(19) United States
(12) Patent Application Publication
(10) Pub. No.: US 2026/0144452 A1
(43) Pub. Date: May 28, 2026

(54) SYSTEMS AND METHODS FOR DETECTING IMPLANT FAILURES USING SMART IMPLANTS WITH SENSORS

(71) Applicant: INDUS, INC., San Jose, CA (US)

(72) Inventors: Saiprasad Sankaranarayanan, San Jose, CA (US); Aditya Dhara, Sunnyvale, CA (US)

(21) Appl. No.: 19/399,437

(22) Filed: Nov. 24, 2025

Related U.S. Application Data
(60) Provisional application No. 63/724,163, filed on Nov. 22, 2024.

(51) Int. Cl. A61B 5/07; A61B 5/00; A61B 17/04

(52) U.S. Cl. CPC A61B 5/076; A61B 5/0031; A61B 17/0401; A61B 2017/0414; A61B 2017/044; A61B 2017/0458

(57) ABSTRACT

A system for monitoring a medical implant in a subject. The system includes an implantable device and a suture designed to affix the implantable device to a surgery site. The device includes a housing comprising an internal cavity, a power source disposed in the internal cavity, a transceiver disposed in the internal cavity, a sensor disposed in the internal cavity.

### RELATED APPLICATIONS

This application is a utility application of Provisional U.S. Patent Application 63/724,163, filed Nov. 22, 2024, all of which is hereby incorporated by reference.

### FIELD OF DISCLOSURE

The field of the invention relates generally to the field of medicine, medical devices, orthopedics, dental, cardiovascular and general surgery, specifically methods and devices useful for detecting failure of medical implants in subjects.

### BACKGROUND

Implantable medical devices have become integral to modern healthcare, offering solutions for structural support, drug delivery, and physiological monitoring. Common examples include orthopedic implants, cardiovascular stents, pacemakers, and neurostimulators. These devices are designed to restore function, alleviate symptoms, and improve patient outcomes over extended periods.

Despite their widespread use, the long-term success of implantable devices depends on their ability to maintain structural integrity and functional performance within the body. Current monitoring approaches primarily rely on periodic imaging techniques such as X-rays, CT scans, or MRI, as well as clinical assessments based on patient-reported symptoms. These methods are inherently intermittent and often fail to detect early signs of implant degradation, loosening, or failure. As a result, complications such as infection, mechanical wear, corrosion, and material fatigue may progress unnoticed until they reach a critical stage, necessitating revision surgery or causing adverse health outcomes.

Existing implantable devices that incorporate sensing capabilities are typically limited to monitoring their own operational parameters, such as battery life or electrical performance in pacemakers. They do not provide comprehensive information about the condition of other implants within the same patient. This lack of inter-device communication and systemic monitoring creates gaps in postoperative care, particularly for patients with multiple implants or complex surgical histories.

Furthermore, current solutions often require external diagnostic equipment and clinical intervention to assess implant health. These approaches increase healthcare costs, impose logistical burdens on patients, and delay timely detection of complications. There is a growing need for continuous, real-time monitoring systems that can provide actionable insights into implant performance and surrounding tissue conditions without relying solely on scheduled clinical evaluations.

### SUMMARY

The present systems, methods, and apparatus overcome many of the shortcomings and limitations of the prior art devices and systems discussed above. Briefly described, aspects of the present disclosure generally relate to systems, methods, and apparatuses and include several embodiments of a smart sensor designed to detect motion and monitor the status of an implanted device in a body. The present disclosure provides systems and methods for detecting motion and monitoring the status of an implant device within a subject's body, particularly after a surgical procedure has been performed. The system utilizes a biocompatible implant device that can be either embedded in or attached to medical implants, or placed inside the human body. This implant device continuously collects and transmits a range of data, such as the spatial position, suture strain, suture resistance, pH, temperature, and other parameters critical to the health and performance of the implant at the surgical site. The data is transmitted to an external monitoring device or software tool, which analyzes the data to detect any discrepancies indicating potential failure of the implant.

In one aspect, the system provides an implantable medical device that is capable of continuously collecting and transmitting data 24/7. The implant device transmits data regarding the 3D spatial position of the implant, relative to other implants and to an external transceiver. Additionally, the implant device tracks changes in strain or resistance on the sutures or other fixation devices used to secure the implant, and it monitors critical parameters like pH and temperature at the implant site. This data is transmitted to an external monitor device for real-time evaluation.

In another aspect, the system also includes innovative sensor materials incorporated into the sutures used to secure the implants. The sutures have multiple layers, each serving a distinct function in facilitating data transmission and monitoring. The first layer is made of a conductive material that measures strain, resistance, and other mechanical changes. Suitable conductive materials include, but are not limited to, conductive rubbers, conductive polymers (e.g., polypyrrole, polyaniline), carbon-based materials (e.g., carbon nanotubes, graphene), metallic wires, and silver or copper-based filaments. The second layer surrounding the conductive material serves as an insulating layer to prevent electrical short circuits and interference from surrounding tissue. Suitable insulating materials may include biocompatible polymers such as polyimide, polyurethane, or silicone-based materials. This insulation ensures that the signal transmission remains stable and unaffected by environmental factors in the body. The third layer is designed to transmit information between the implants. This layer can be made from a material that supports electromagnetic transmission or a wireless communication protocol such as Bluetooth, near-field communication (NFC), or RF. Materials that could serve this function include polymer-based materials integrated with antennas or other wireless components capable of transmitting data to external devices. This multi-layered suture technology provides a unique advantage in continuously monitoring the condition of the implants and the surrounding tissue, ensuring that any mechanical failure, such as changes in the relative position or strain of the implant, can be detected early. Moreover, the sensor technology integrated into the sutures is biocompatible, biodegradable, and capable of offering real-time feedback to healthcare providers, enhancing the overall success of the implant and recovery process.

In some embodiments, the implant device is integrated into other medical implants during the surgical procedure. The implant device may be embedded within the implant or attached externally on the implant. The implant device is designed to provide valuable insights into the implant's operational status, including the physical and chemical conditions surrounding the implant, to ensure early detection of any signs of malfunction or failure.

In some embodiments, the implant device may be made from biodegradable materials or materials designed to degrade after a predetermined period, such as six months, depending on the intended use of the implant. The sensor device may degrade through morphological changes, chemical reactions, or other processes, with the degradation potentially releasing drugs or bioactive compounds that aid in tissue regeneration around the implant.

In some embodiments, the implants monitored by this system may include, but are not limited to, orthopedic implants such as joint replacements, plates, screws, rods (intramedullary and extramedullary), spinal rods, bone anchors, as well as biological implants like transplanted organs, allografts, xenografts, and various prosthetics or orthotics. The sensor system is designed to function effectively with these various implant types to ensure the long-term success of the surgical procedure and the wellbeing of the patient.

In some embodiments, the implant device is made of biodegradable materials. In some embodiments, the implant device is designed to degrade in the body over a predetermined period of time. For example, the device is installed during a surgical procedure that requires implants to be stable for a period of six months, and the device degrades after a period of six months.

In some embodiments, the implant devices are orthopedic implants including total joint replacement implants, plates and screws, intramedullary and extramedullary rods, autografts, xenografts and allografts, external fixators, internal fixators, cement, spinal rods, metallic and non-metallic bone anchors, orthotics and prosthesis. In some embodiments, the implants are biological implants including but not limited to transplanted organs, allografts, xenografts, other implanted tissues.

In some embodiments, the sensor device is made of biomaterials that help in regeneration of cells and tissues surrounding the implant. In some embodiments, the degradation of the sensor device leads to release of drugs and bioactive compounds to the surrounding tissues.

In one aspect, the system provides a method for detecting motion of implant devices in subjects, wherein the motion of the implant devices is detected using a sensor that is fitted with the implant devices.

In some embodiments, electromagnetic waves are transmitted to the implant device to be received back, in order to identify the position, coordinates, angles, and distance of the sensor device.

In some embodiments, the external monitor device is a mobile phone. In some embodiments, the external monitor device is a personal computer. In some embodiments, the external monitor device connects to other devices such as personal computers and mobile phones using wireless connections.

Other objects, features and advantages of the present system will become apparent from the following detailed description. It should be understood, however, that the detailed description and the specific examples, while indicating specific embodiments of the system, are given by way of illustration only, since various changes and modifications within the spirit and scope of the system will become apparent to those skilled in the art from this detailed description

In one aspect, a system for monitoring a medical implant in a subject is provided. The system includes an implantable device, the device including a housing comprising an internal cavity, a power source disposed in the internal cavity, and a transceiver disposed in the internal cavity, a sensor disposed in the internal cavity designed to collect, measure, or monitor a parameter of the subject or the medical implant, a suture, designed to affix the implantable device to a surgery site, and an external device, wherein the external device is in electronic communication with the transceiver and the sensor.

In some instances, the implantable device further comprises an eyelet.

In other instances, the suture is designed to be disposed through the eyelet.

In yet further instances, the sensor is in electronic communication with the transceiver.

In some instances, the parameter includes the implant's spatial position, a suture strain, a suture resistance, pH, temperature, or any combination thereof.

In other instances, the external device is imparted with a predetermined threshold or a predetermined baseline.

In yet further instances, the external device is compares one or more measurements to the predetermined threshold or the predetermined baseline.

In some instances, the external device is a portable electronic device or a computer.

In other instances, the sensor is designed to track a three-dimensional spatial position of the medical implant in the subject.

In yet further instances, the transceiver is designed to receive electronic communications from the external device.

In another aspect, a system for detecting a failure of an implanted device is provided. The system includes a biocompatible sensor designed to be imbedded in a surgical site of a subject and wherein the biocompatible sensor collects one or more measurements related to a parameter and a communication module designed to be in electronic communication with the biocompatible sensor. The biocompatible sensor transmits the one or more measurements to the communication module and the communication module evaluates the one or more measurements transmitted.

In some instances, the communication module is imparted with a predetermined threshold or predetermined baseline.

In other instances, the communication module compares the one or more measurements to the predetermined threshold or the predetermined baseline.

In yet further instances, the biocompatible sensor comprises a multi-layer suture, the suture includes a conductive layer designed to collect the one or more measurements related to the parameter, an insulating later designed to surround the conductive layer and prevent electrical interference, and a transmission layer configured to provide wireless communication between the conductive layer and the communication module.

In some instances, the biocompatible sensor includes a housing and a suture designed to secure the housing to the surgical site, where the housing includes a power source, a transceiver, and a sensor.

In yet another aspect, a method for monitoring an implanted device is provided. The method includes providing a biocompatible sensor, inserting the biocompatible sensor into a surgical site, measuring a parameter of the surgical site, transmitting the parameter from the biocompatible sensor to an external monitoring device, comparing the parameter to a predetermined baseline or a predetermined threshold, and generating an alert on the external monitoring device.

In some instances, the biocompatible sensor comprises a multi-layer suture, where the suture includes a conductive layer designed to collect the one or more measurements related to the parameter, an insulating later designed to surround the conductive layer and prevent electrical interference, and a transmission layer configured to provide wireless communication between the conductive layer and the communication module.

In another instance, the biocompatible sensor includes a housing and a suture designed to secure the housing to the surgical site, where the housing comprises a power source, a transceiver, and a sensor.

In yet another instances, the method further includes tracking a three-dimensional spatial position of the biocompatible sensor in the surgical site.

In some instances, the method further includes tracking a three-dimensional spatial position of a second implanted device.

These and other aspects and advantages of the present disclosure will become apparent to those skilled in the art after considering the following detailed description in connection with the accompanying drawings.

### BRIEF DESCRIPTION OF THE DRAWINGS

FIG. 1A is side view of an implant detection device, according to the disclosure herein;

FIG. 1B is a partial cross-sectional view of the implant detection device of FIG. 1A;

FIG. 2A is a side view of an implant detection device, according to the disclosure herein;

FIG. 2B is a partial cross-sectional view of the implant detection device of FIG. 2A;

FIG. 3 is a perspective view of an implantable implant failure detection device, according to the disclosure herein;

FIG. 4 is a cross-sectional view of a surgery site, showing an implant detection device according to the disclosure herein, with the implant detection device including a tissue-facing eyelet;

FIG. 5 is a cross-sectional view of a surgery site, showing an implant detection device according to the disclosure herein, with the implant detection device including a bone-facing eyelet;

FIG. 6 is a cross-sectional view of a suture, according to the disclosure herein;

FIG. 7 is a flowchart including a method for monitoring an implanted device;

### DETAILED DESCRIPTION

Reference will now be made in detail to the presently preferred embodiments of the system which, together with the drawings and the following examples, serve to explain the principles of the system. These embodiments are described in sufficient detail to enable those skilled in the art to practice the system, and it is understood that other embodiments may be utilized, and that structural, biological, and chemical changes may be made without departing from the spirit and scope of the present system. Unless defined otherwise, all technical and scientific terms used herein have the same meanings as commonly understood by one of ordinary skills in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

“Apparatus” and “device” are used interchangeably herein.

As used herein, the term “about” means plus or minus 10% of the numerical value of the number with which it is being used.

Before any aspects are described in detail, it is to be understood that the disclosure is not limited in its application to the details of construction and the arrangement of components set forth in the following description or illustrated in the following drawings, which is limited only by the claims that follow the present disclosure. The disclosure is capable of other aspects, and of being practiced, or of being carried out, in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following description is presented to enable a person skilled in the art to make and use aspects of the disclosure. Various modifications to the illustrated aspects will be readily apparent to those skilled in the art, and the generic principles herein can be applied to other aspects and applications without departing from aspects of the disclosure. Thus, aspects of the disclosure are not intended to be limited to aspects shown but are to be accorded the widest scope consistent with the principles and features disclosed herein. The following detailed description is to be read with reference to the figures, in which like elements in different figures have like reference numerals. Skilled artisans will recognize the examples provided herein have many useful alternatives and fall within the scope of aspects of the disclosure.

Additionally, while the following discussion may describe features associated with specific devices or aspects, it is understood that additional devices and/or features can be used with the described systems and methods, and that the discussed devices and features are used to provide examples of possible aspects, without being limited.

In one embodiment, the system provides a comprehensive system for monitoring medical implants within a subject's body, designed to enhance the detection of motion, strain, and other critical parameters that can indicate potential issues with the implant.

In some embodiments, this system utilizes a biocompatible implant device that can either be embedded into or attached to medical implants.

In some embodiments, the sensor is capable of continuously collecting and transmitting a variety of data, which is essential for ensuring the proper functioning of the implant post-surgery.

In some embodiments, the data collected includes parameters such as the implant's spatial position, suture strain, suture resistance, pH, temperature, and other critical factors that affect the health and performance of the implant.

In some embodiments, the disclosure provides a system that identifies movement in implants using a sensor device, wherein the sensor device reflects and transmits electromagnetic waves to an external monitor device that evaluates collected data over a period of time.

In some embodiments, the sensor is a core component of the system, capable of working around the clock to monitor the status of the implant.

In some embodiments,, the sensor tracks the 3D spatial position of the implant relative to other implants and to an external transceiver. This feature is crucial for monitoring any displacement or misalignment of the implant over time, as such shifts can indicate potential failure or complications.

In some embodiments, the sensor measures strain or resistance experienced by the sutures or other fixation devices that secure the implant in place. Monitoring changes in strain is essential because it can provide early indications of loosening or failure of the fixation system.

In some embodiments, parameters, such as pH and temperature, are also monitored at the implant site. These factors are significant because changes in pH or temperature can indicate infection, inflammation, or other adverse conditions around the implant.

In some embodiments, the system introduces innovative sensor technology integrated into the sutures used to secure medical implants. These sutures have multiple layers, each with a distinct purpose that contributes to data transmission and monitoring.

In some embodiments, one layer of the suture is made of a conductive material, which is responsible for measuring mechanical changes such as strain, resistance, and other factors that affect the integrity of the implant and the surrounding tissue. Suitable materials for this conductive layer include conductive rubbers, conductive polymers (e.g., polypyrrole, polyaniline), carbon-based materials (e.g., carbon nanotubes, graphene), metallic wires, and filaments made of silver or copper.

In some embodiments, one layer surrounding the conductive material serves as insulation, preventing electrical short circuits and interference from surrounding tissue. This insulating layer is made from biocompatible materials such as polyimide, polyurethane, or silicone-based compounds. It ensures that the signals generated by the conductive layer are stable and reliable, unaffected by the body's complex internal environment.

In some embodiments, one layer is designed to facilitate data transmission between implants. This layer can support wireless communication protocols, such as Bluetooth, near-field communication (NFC), or RF. Materials such as polymer-based composites integrated with antennas or wireless components are used to enable the transmission of data from the implant to external monitoring devices. This multi-layered suture design ensures that the system provides continuous monitoring of the implant, detecting any mechanical issues or changes in the surrounding tissue that may signal failure.

In certain embodiments, the sensor device is made from biodegradable materials that degrade after a predetermined period, depending on the specific use case of the implant. For example, in cases where the implant requires a stable environment for six months, the sensor device may degrade gradually over that period. This degradation could occur through morphological changes, chemical reactions, or other biological processes within the body. As the sensor degrades, it may release drugs or bioactive compounds that promote tissue regeneration around the implant, enhancing recovery and ensuring the long-term success of the implant. The materials used for the sensor are chosen not only for their ability to degrade over time but also for their biocompatibility. This ensures that the sensor can safely function within the body without causing adverse reactions. Additionally, the degradation process can be designed to occur gradually, ensuring that the sensor does not interfere with the implant's function during the critical early recovery phase.

The sensor system described in the disclosure is versatile and can be used with a variety of medical implants. Some of the implants that can benefit from this technology include orthopedic implants such as total joint replacements, plates, screws, rods (both intramedullary and extramedullary), bone anchors, and spinal rods. The system is also applicable to biological implants, such as transplanted organs, allografts, xenografts, and other implanted tissues. In these cases, the sensor system helps monitor the implant's position, condition, and the health of surrounding tissue, ensuring that any potential issues are detected early.

In some embodiments, the sensor system is integrated into the implant during the surgical procedure. The sensor can be embedded directly within the implant or attached externally, depending on the design and type of implant. Once the sensor is in place, it begins transmitting data to the external monitoring device, providing continuous feedback to the healthcare team about the status of the implant and the surrounding tissues.

In some embodiments, a medical professional places one or more implant devices into a human body during a surgical procedure.

In some embodiments, the implants are orthopedic implants including total joint replacement implants, plates and screws, intramedullary and extramedullary rods, autografts, xenografts and allografts, external fixators, internal fixators, cement, spinal rods, metallic and non-metallic bone anchors, orthotics and prosthesis.

In some embodiments, the implants are biological implants including but not limited to transplanted organs, allografts, xenografts, other implanted tissues.

In some embodiments, the implant device reflects and transmits electromagnetic waves to an external monitor device that evaluates collected data over a period of time.

In some embodiments, the implant device is made of biodegradable materials.

In some embodiments, the sensor device is made of biomaterials that help in regeneration of cells and tissues surrounding the implant.

In some embodiments, the degradation of the sensor device leads to release of drugs and bioactive compounds to the surrounding tissues.

In some embodiments, the implant device is placed inside a cavity in other implantable devices.

In some embodiments the sensor device is made of materials that can be sterilized.

In some embodiments the sensor device may be made of bio-absorbable material.

In some embodiments the sensor device may be made of metal or plastic.

In some embodiments, the sensor device reflects electromagnetic waves.

In some embodiments, the sensor device communicates with an external monitor device through the transmission of electromagnetic waves. For example: Radio waves could be transmitted to achieve wireless non-contact transfer of data.

In some embodiments, the sensor device is in a combination of shapes, continuous, or discontinuous. The list of possible shapes includes, but is not limited to, circles, triangles, rectangles, squares, rhomboids, trapezoids, and any other regular or irregular polygons.

In some embodiments, the implant device is in the shape of a four sided figure with surface area ranging from 2.5×10−7 cm2 to 400 cm2 .

The term “subject” as used herein is not limiting and is used interchangeably with patient. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like.

In some embodiments, the implant device is comprised of materials selected from the group consisting of Stainless steel 316L, Cobalt-Chrome-Molybdenum, Cobalt, Chromium, Molybdenum, Nickel, Titanium, Aluminum, Vanadium, Iron, Yttrium, Niobium, Zirconium, Hydroxyapatite-coated Titanium, Partially stabilized zirconia, Poly methacrylic acid, Polyethylene, Poly Dimethyl Sulphoxide, Polypropylene, Polysulfone, Polycarbonate, Polyglycolic acid, Polylactic acid, Polycaprolactone, Polydioxanone, Polysulfates, Polyphosphates, Polyhydroxyalkanoates, Polyphenols, Silk fibroin, Collagen, Hydrogel, Keratin, Elastin, Rubber Latex, Polysaccharides, Chitin, Chitosan, Pullulan, Calcium Phosphate, Dicalcium phosphate, Dicalcium phosphate dihydrate, Octacalcium phosphate, Tricalcium phosphate, Tetracalcium phosphate, Conductive rubbers, Conductive polymers (e.g., polypyrrole, polyaniline), Carbon-based materials (e.g., carbon nanotubes, graphene), Metallic wires, Silver-based filaments, Copper-based filaments.

The system will now be described with reference to the drawing figures, in which like reference numerals refer to like parts throughout. For purposes of clarity in illustrating the characteristics of the present system, proportional relationships of the elements have not necessarily been maintained in the drawing figures.

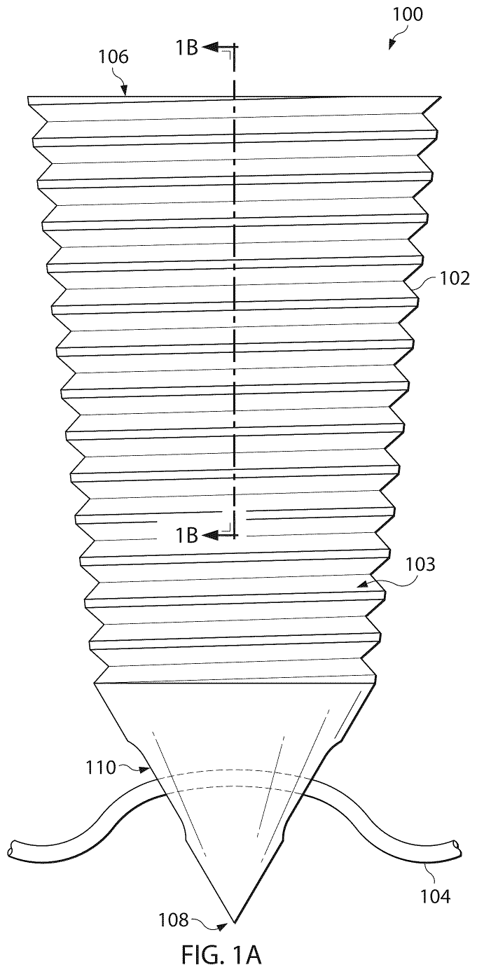

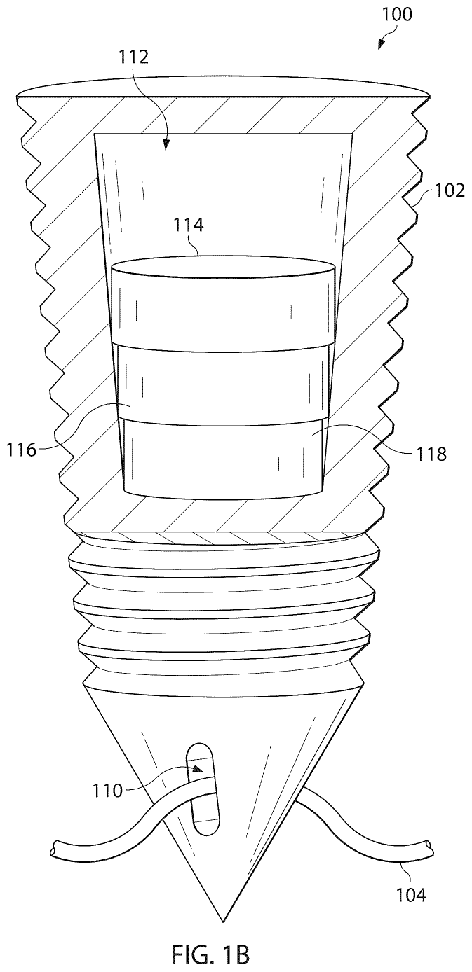

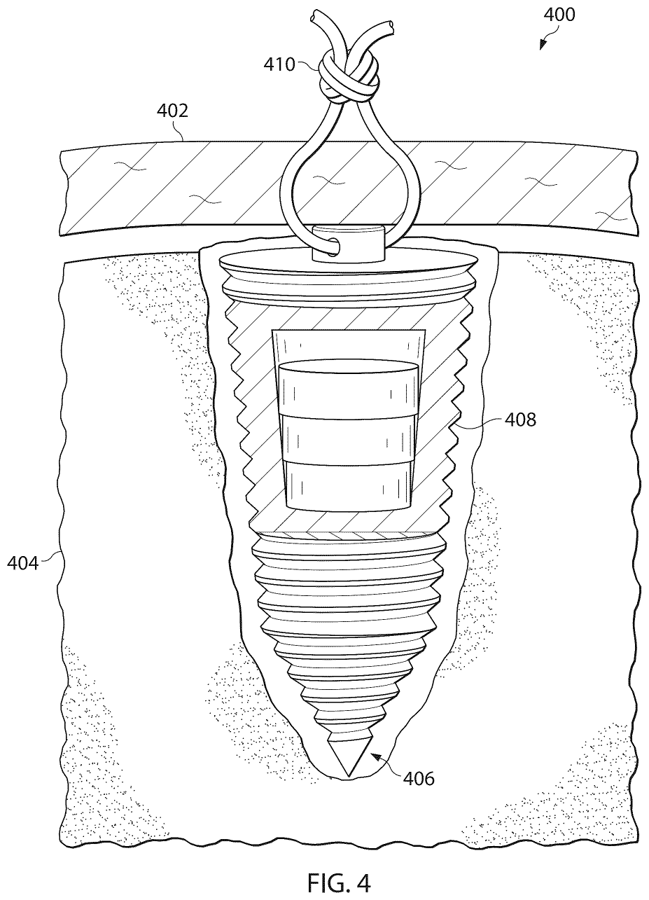

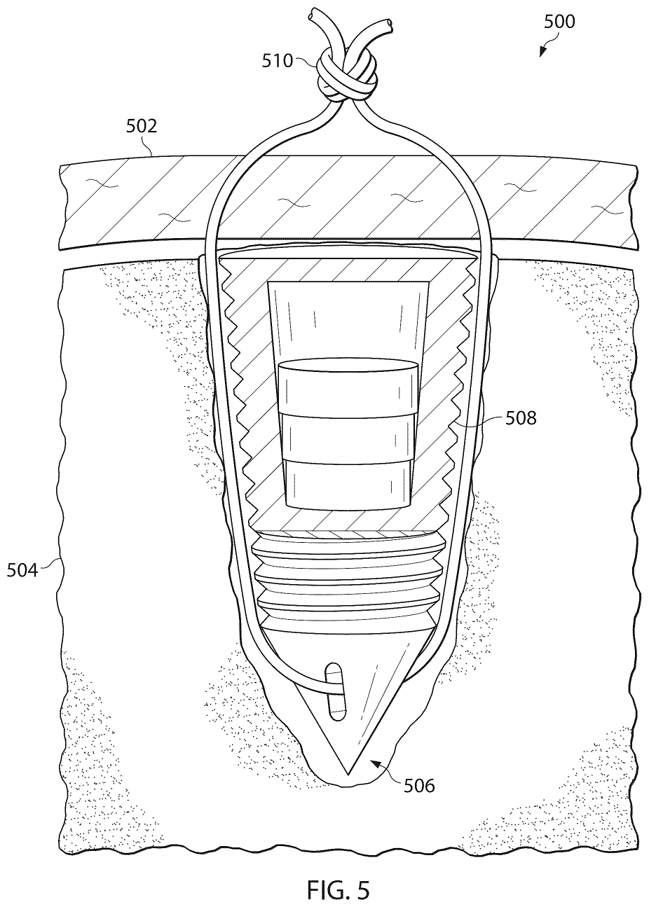

Turning now to FIGS. 1A and 1B, one embodiment of an implant monitoring device 100 in according to the principles of this disclosure, is provided. The implant monitoring device may be designed to monitor medical implants within a subject's body and detect motion, strain, and other parameters that may indicate potential issues with a medical implant. The implant monitoring device 100 comprises a body 102 and a suture 104. The body 102 may be designed to house one or more sensors, chips, processors, or monitoring devices. The suture 104 may be designed to secure the implant monitoring device 100 to an implantation site (such as in FIGS. 4 and 5).

The body 102 may have a tissue facing end 106 and a bone facing end 106. At the bone facing end 108, the body 102 may further comprise an eyelet 110. The eyelet 110 may be designed such that the suture 104 can be threaded through the eyelet 110 in order to secure the implant monitoring device to the implantation site.

The body 102 may further comprise an internal cavity 112. In some instances, the internal cavity 112 may include a battery 114, a control system 116, and a transceiver 118. The battery 114 may be designed to supply power to the control system 116 and the transceiver 118.

The control system 116 may further include at least one sensor, where the sensor is designed to collect, measure, or monitor a particular parameter of the subject. In some instances, the parameter may include the implant's spatial position, suture strain, suture resistance, pH, temperature, and other factors that affect the health and performance of the implant. In other instances, the sensor may track the three-dimensional spatial position of the implant relative to other implants and to an external transceiver (not depicted). In yet further instances, the sensor measures strain or resistance experienced by the suture 104 or other fixation devices (not depicted) that secure the implant 100 in place.

Still referring to FIG. 1A and FIG. 1B, the control system 116 may be in communication with an external user device (not depicted). The external user device may be provided in the form of a cell phone, tablet, laptop computer, desktop computer, or any other similar electronic device that may include a camera and a user interface. The external user device may include native, mobile or web-based applications to facilitate user communication with the sensor 116 or the transceiver 118.

In some instances, machine learning (ML), artificial intelligence (AI), or similar processes may be implemented to iteratively train the device 100 and improve performance of the device 100 based on one or more feedback parameters, characteristics, or similar information. For example, in some instances, ML/AI may be used to predict when a parameter of an implantable device is going to exceed or a predetermined baseline or a predetermined threshold, such that a surgeon can intervene before failure of the implantable device occurs.

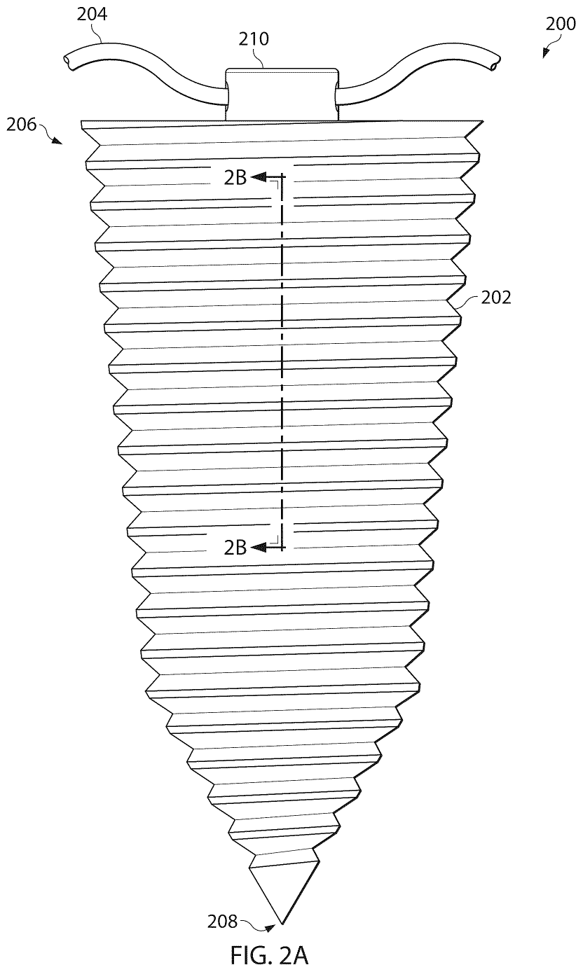

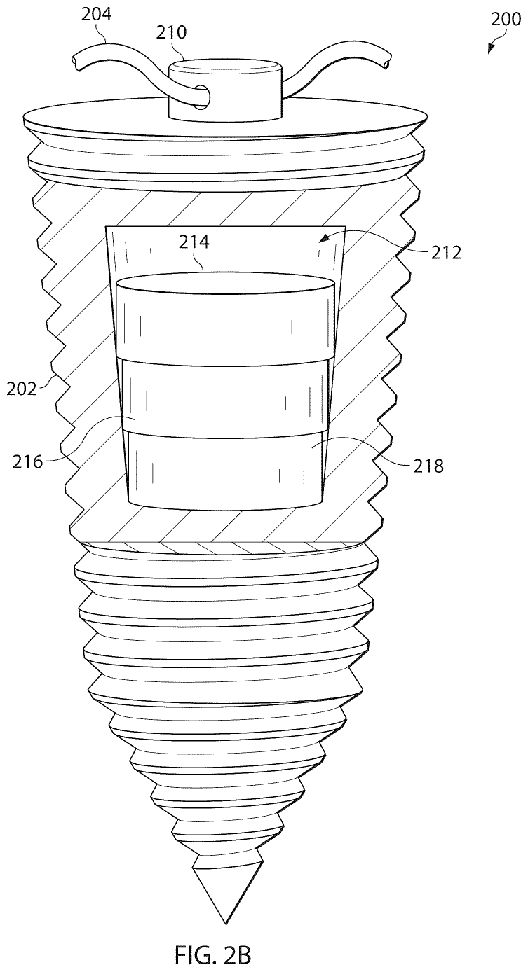

Turning to FIGS. 2A and 2B, one embodiment of an implant monitoring device 200 in according to the principles of this disclosure is shown. The implant monitoring device may be substantially the same as the implant monitoring device 100 of FIG. 1A-1B. The implant monitoring device may be designed to monitor medical implants within a subject's body and detect motion, strain, and other parameters that may indicate potential issues with a medical implant. The implant monitoring device 200 comprises a body 202 and a suture 204. The body 202 may be designed to house one or more sensors, chips, processors, or monitoring devices. The suture 204 may be designed to secure the implant monitoring device 200 to an implantation site (such as in FIGS. 4 and 5).

The body 202 may have a tissue facing end 206 and a bone facing end 206 at the bone facing end 206. The body 202 may further comprise an eyelet 210 disposed on the tissue facing end 206 of the body 202. The eyelet 210 may be designed such that the suture 204 can be threaded through the eyelet 210 in order to secure the implant monitoring device to the implantation site.

The body 202 may further comprise an internal cavity 212. In some instances, the internal cavity 212 may include a battery 214, a control system 216, and a transceiver 218. The battery 214 may be designed to supply power to the control system 216 and the transceiver 218.

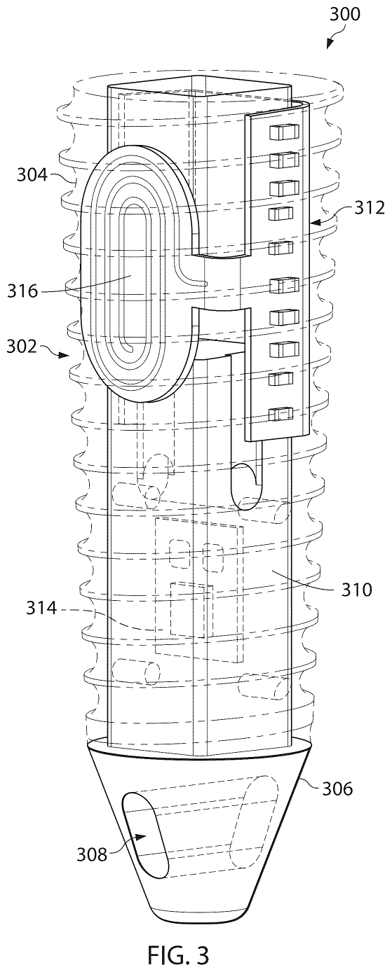

Turning to FIG. 3, one embodiment of an implant monitoring device 300 in according to the principles of this disclosure, is provided. The implant monitoring device 300 may be substantially similar to the implant monitoring device 100 of FIGS. 1A and 1B or the implant monitoring device 200 of FIGS. 2A and 2B. The implant monitoring device 300 may include a body 302, where the body 302 is provided with one or more threads 304. The body 302 may include an insertion end 306. The insertion end 306 may be designed to aid in the implantation and securement of the device 100. In some embodiments, the insertion end 306 may comprise a generally conical shape or any other suitable shape without departing from the principles of this disclosure. The insertion end 306 may further include an eyelet 308. The eyelet 308 may be designed to receive a suture (not depicted) as described about with respect to the implant monitoring devices 100, 200 shown in FIGS. 1-2.

The body 302 may define an internal cavity 310. The internal cavity 310 may be designed to house at least one printed circuit board 312. The printed circuit board 312 may include a battery or any other suitable electronics and, in some embodiments, a near field communication device 316, wherein the near field communication device may be designed to be in communication with external devices.

The internal cavity 310 may further house at least one sensor 314. Sensor 314 may be substantially the same as the sensors described above with respect to FIG. 1-2. Sensor 314 may comprise a strain sensor, a pH sensor, a temperature sensor, or any combination thereof. The sensor 314 may be in communication with the suture (not depicted) that affixes the device 300 to a surgical site (not depicted). Of course, sensor 314 may comprise additional sensors without departing from the principles of this disclosure.

Turning to FIG. 4, an exemplary surgical implantation site 400 is provided, where a device may be anchored. The site includes tissue 402 and bone 404. The bone 404 may define a cavity 406. The device 200 of FIGS. 2A and 2B may be inserted into the cavity 406, such that the eyelet 210 is positioned near an opening 408 of the cavity 406. The suture 204 may be sutured through the eyelet 210 and the tissue 402, such that the device 200 is secured within the implantation site 400.

Turning to FIG. 5, an exemplary surgical implantation site 500 is provided. The site includes tissue 502 and bone 504. The bone 504 may define a cavity 506. The device 100 of FIGS. 1A and 1B may be inserted into the cavity 506, such that the eyelet 110 is positioned near a base 508 of the cavity 506. The suture 104 may be inserted through the eyelet 110 and sutured through the tissue 502, such that the device 100 is secured within the implantation site 500.

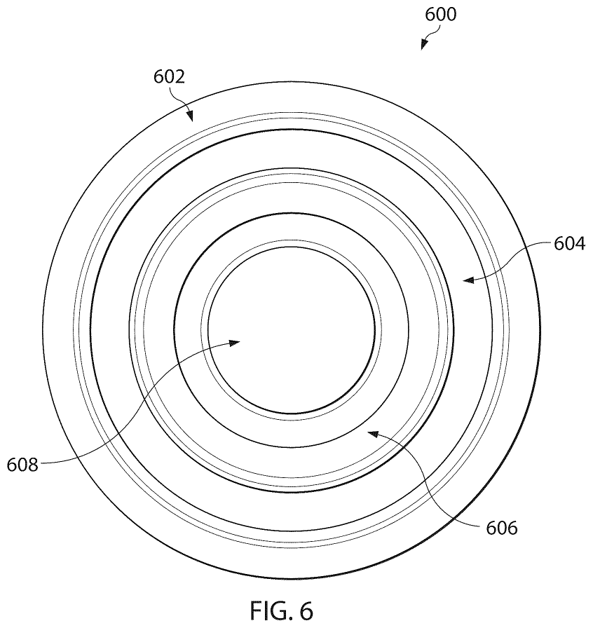

Turning to FIG. 6, a cross-section of one embodiment of a suture 600 in according to the principles of this disclosure, is provided. The suture 600 may be substantially the same as the suture 104 of FIG. 1A-1B or the suture 204 of FIG. 2A-B. The suture 600 may include an insulating layer 602 and an embedded sensor 604. The insulating layer 602 may mostly, substantially, or completely surround the embedded sensor 604. The sensor 604 may be substantially the same as any sensor described herein. The insulating layer 602 may be designed to prevent electrical short circuits and interference with any surrounding tissue near the suture 600. The insulating layer may be provided in the form of biocompatible materials such as polyimide, polyurethane, or silicone-based compounds.

The sensor 604 may be designed to measure, monitor, or detect changes in one or more parameters of an implantable device. The sensor 604 may be provided in the form of a conductive matter such as conductive rubbers, conductive polymers (e.g., polypyrrole, polyaniline), carbon-based materials (e.g., carbon nanotubes, graphene), metallic wires, and filaments made of silver or copper, or any combination thereof.

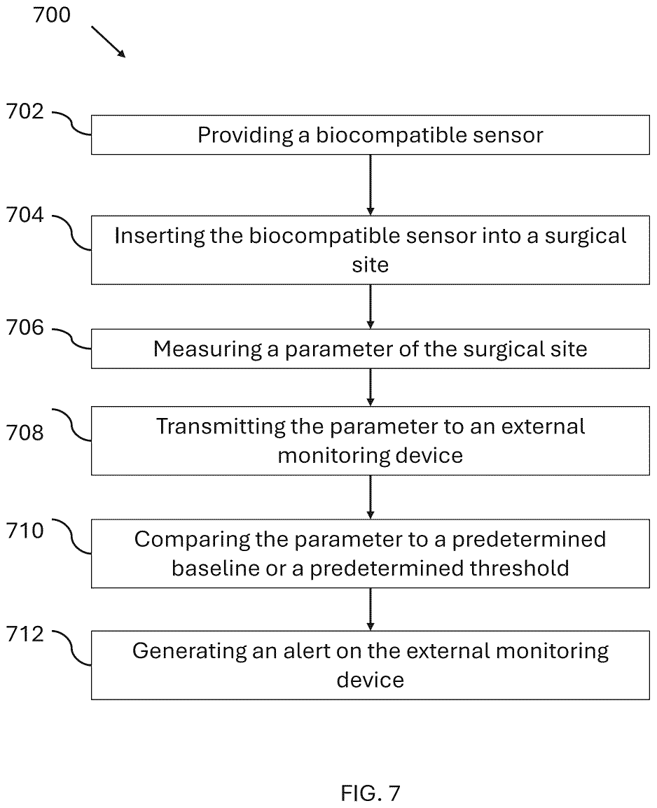

Turning to FIG. 7, one embodiment of a method for monitoring an implanted device 700 in according to the principles of this disclosure, is provided. At 702, the method 700 includes providing a biocompatible sensor. At 704, the method 700 includes inserting the biocompatible sensor into a surgical site. At 706, the method 700 includes measuring a parameter of the surgical site. At 708, the method 700 includes transmitting the parameter to an external monitoring device. At 710, the method 700 includes comparing the parameter to a predetermined baseline or a predetermined threshold. At 712, the method 700 includes generating an alert on the external monitoring device. In some embodiments, the alert generated at step 712 may occur because the parameter measured falls below the baseline or outside the predetermined threshold. The alert generated at step 712 may be visual, auditory, or any other suitable form without departing from the principles of this disclosure.

It will be appreciated by those skilled in the art that while the above disclosure has been described above in connection with particular aspects and examples, the above disclosure is not necessarily so limited, and that numerous other aspects, examples, uses, modifications and departures from the aspects, examples and uses are intended to be encompassed by the claims attached hereto. The entire disclosure of each patent and publication cited herein is incorporated by reference, as if each such patent or publication were individually incorporated by reference herein. Various features and advantages of the above disclosure are set forth in the following claims.

What is claimed is:

1. A system for monitoring a medical implant in a subject, the system comprising:
an implantable device, the device comprising:
a housing defining an internal cavity;
a power source disposed in the internal cavity;
a transceiver disposed in the internal cavity;
a sensor disposed in the internal cavity, the sensor designed to collect,
measure, or monitor a parameter of the subject or the medical implant;
a suture configured to affix the implantable device to a surgery site; and
an external device in electronic communication with the transceiver and the sensor.

2. The system of claim 1, wherein the implantable device further comprises an eyelet.

3. The system of claim 2, wherein the suture is designed to be disposed through the eyelet.

4. The system of claim 1, wherein the sensor is in electronic communication with the transceiver.

5. The system of claim 1, wherein the parameter includes the implant's spatial position, a suture strain, a suture resistance, pH, temperature, or any combination thereof.

6. The system of claim 1, wherein the external device is imparted with a predetermined threshold or a predetermined baseline.

7. The system of claim 6, wherein the external device is compares one or more measurements to the predetermined threshold or the predetermined baseline.

8. The system of claim 1, wherein the external device is a portable electronic device or a computer.

9. The system of claim 1, wherein the sensor is designed to track a three-dimensional spatial position of the medical implant in the subject.

10. The system of claim 1, wherein the transceiver is designed to receive electronic communications from the external device.

11. A system for detecting a failure of an implanted device, the system comprising:
a biocompatible sensor designed to be imbedded in a surgical site of a subject, the biocompatible sensor configured to collect one or more measurements related to a parameter; and
a communication module designed to be in electronic communication with the biocompatible sensor,
wherein the biocompatible sensor transmits the one or more measurements to the communication module and wherein the communication module evaluates the one or more measurements transmitted.

12. The system of claim 11, wherein the communication module is imparted with a predetermined threshold or predetermined baseline.

13. The system of claim 12, wherein the communication module compares the one or more measurements to the predetermined threshold or the predetermined baseline.

14. The system of claim 11, wherein the biocompatible sensor comprises a multi-layer suture, the suture comprising:
a conductive layer designed to collect the one or more measurements related to the parameter;
an insulating later designed to surround the conductive layer and prevent electrical interference; and
a transmission layer configured to provide wireless communication between the conductive layer and the communication module.

15. The system of claim 11, wherein the biocompatible sensor comprises:
a housing; and
a suture designed to secure the housing to the surgical site,
wherein the housing comprises a power source, a transceiver, and a sensor.

16. A method for monitoring an implanted device, comprising:
providing a biocompatible sensor;
inserting the biocompatible sensor into a surgical site;
measuring a parameter of the surgical site;
transmitting the parameter from the biocompatible sensor to an external monitoring device;
comparing the parameter to a predetermined baseline or a predetermined threshold; and
generating an alert on the external monitoring device.

17. The method of claim 16, wherein the biocompatible sensor comprises a multi-layer suture, the suture comprising:
a conductive layer designed to collect the one or more measurements related to the parameter;
an insulating later designed to surround the conductive layer and prevent electrical interference; and
a transmission layer configured to provide wireless communication between the conductive layer and the communication module.

18. The method of claim 16, wherein the biocompatible sensor comprises;
a housing; and
a suture designed to secure the housing to the surgical site,
wherein the housing comprises a power source, a transceiver, and a sensor.

19. The method of claim 16, the method further comprising tracking a three-dimensional spatial position of the biocompatible sensor in the surgical site.

20. The method of claim 16, the method further comprising tracking a three-dimensional spatial position of a second implanted device.
